(19) United States
(12) Patent Application Publication
(10) Pub. No.: US 2009/0291061 A1
(43) Pub. Date: Nov. 26, 2009

(54) STEM CELL THERAPY FOR BLOOD VESSEL DEGENERATION

(76) Inventors: Neil H. Riordan, Chandler, AZ (US); Thomas E. Ichim, San Diego, CA (US)

Correspondence Address: BAUMGARTNER PATENT LAW, 5933 N.E. WIN SIVERS DR. SUITE 250, PORTLAND, OR, 97220, US

(21) Appl. No.: 12/470,438

(22) Filed: May 21, 2009

Related U.S. Application Data
(60) Provisional application No. 61/055,106, filed on May 21, 2008.

(51) Int. Cl. A61K 38/20; A61K 45/00; A61P 9/00

(52) U.S. Cl. 424/85.2; 424/93.7

(57) ABSTRACT

The present disclosure provides means of treating degenerated blood vessels through administration of stem cells or activators of stem cells. In one particular embodiment vessel reactivity is increased through administration of stem cells or stem cell activating compounds. Other embodiments include “reconditioning” of vessels prone to aneurysms, repairing aneurysms of vessels, or acceleration of endothelialization after stent placement. Provided within the invention are methods of rejuvenating properties of said vessels associated with physiological health, examples of which include appropriate production of anti-coagulating/clotting factors, control of angiogenesis, and appropriate revascularization of injured tissue.

### CROSS-REFERENCE TO RELATED APPLICATIONS

This application claims priority to co-pending U.S. Provisional Application Ser. No. 61/055,106, filed May 21, 2008 which is expressly incorporated by reference in its' entirety.

### FIELD OF THE INVENTION

The invention is related to the area of vascular biology, more particularly the invention relates to stimulation of blood vessel regeneration through administration of stem cells alone or in combination with agents capable of stimulating stem cells. More particularly the invention deals with methods of treating aneurysms or blood vessels prone to aneurysms.

### BACKGROUND

There are two main types of arteries: elastic and muscular. Elastic arteries are large in nature (>1 cm diameter) while muscular ones are usually smaller (0.1-10 mm). The aorta is an example of an elastic artery, which is capable of distension and elasticity, which is required for it to be able to stretch as a response to each pulse of the heart. Another example of an elastic artery is the pulmonary artery, which delivers hypoxic blood to the lungs. The carotid, subclavian and renal arteries are also considered elastic arteries. Connective tissue is usually present underneath elastic arteries and the tunica media is characterized by the presence of numerous elastic lamella. In general the elastic arteries are close to the large pressures of the heart and therefore require elastic capabilities to buffer the pulse. The adventitia of the large vessels carries vasa vasorum (small blood vessels feeding the large blood vessel) and nerves. As blood moves away from the large arteries, the medium size arteries are generally muscular in nature. In muscular arteries the tunica media is composed primarily of smooth muscle tissue. The muscular arteries contractile and the extent of contraction or relaxation is governed by endothelium-derived vasoactive substances such as nitric oxide, as well as by the nervous system. Although muscular arteries have some elastic fibers like the elastic arteries, these are not organized into lamella.

The endothelium comprises the lining of blood vessels and is known to actively participate in numerous functions include secretion of coagulation and anti-coagulation factors (1), contraction and relaxation of the blood vessels by elaboration of soluble factors (2), and recruitment of immunocytes (3) and stem cells (4), through expression of adhesion molecules. The major diseases afflicting society are associated with endothelial dysfunction. For example, heart attack and stroke are associated with thrombotic states, usually as a result of hypercoagulation/lack of fibrinolysis. Ischemic heart failure is associated with poor collateralization and angiogenesis. Atherosclerosis, which causes the thickening of the blood vessels leading to ischemia is caused by foam cell accumulation and progression to atheroma. Endothelial dependent migration of monocyte and accessory cells is critical for development of atherosclerosis. Sepsis is caused by endothelial dependent disseminated intravascular coagulation: the only drug for this condition which demonstrated therapeutic benefit, recombinant activated protein C, acts on the endothelium (5). Cancer is also associated with endothelium, in the sense that tumors dependent on endothelium migration and angiogenesis for their growth and metastasis. Accordingly, controlling the endothelium and assuring its health is an important endeavor.

Endothelial damage is caused by numerous factors: In addition to induced conditions endothelial dysfunction, such as smoking, infections, and oxidative stress, endothelial dysfunction also occurs as a natural part of aging. For example, a recent study compared flow mediated dilation responses in healthy patients of various ages, free of cardiovascular risk factors. A statistically significant decline in vasodilatory response was observed that positively correlated with age (6). One of the possible explanations for age-related endothelial dysfunction is decreased ability to secrete the vasoactive small molecule nitric oxide in response to appropriate stimuli. For example, Laurel et al demonstrated inhibited exercise-induced nitric oxide production, and flow mediated dilation in 28 aged (58+/−2) healthy volunteers compared to 29 younger (25+/−1 years old) subjects (7).

Dysfunction of endothelium has been ascribed to numerous possible causes, one of which is low grade inflammation associated with aging. One useful marker of this is plasma levels of C Reactive Protein (CRP). Studies have demonstrated positive correlation between age and plasma CRP (8), as well as CRP and presence of endothelial dysfunction (9). Transgenic expression of CRP in mice leads to endothelial dysfunction, presumably through suppression of nitric oxide production and stimulation of macrophage infiltration into major blood vessels (10).

Dysfunctional and damaged endothelium is known to be replenished by circulating endothelial precursor cells (EPC). It is known that such cells migrate to damaged arterial endothelium as a result of CXCR2 expression on the EPC which response to CXCL1 or CXCL7 secreted by damaged endothelium (11).

Weakening of blood vessels is associated with damaged endothelium, as well as smooth muscle cell apoptosis (12), and disorganization of the extracellular matrix. Certain conditions such as Marfan syndrome predispose to weakening of blood vessels, however senescence and inflammation have been cited causes in the majority of patients. Weakening of blood vessels leads to a variety of circulatory problems, for example aneurysms and aortic dissection.

Aneurysms are blood-filled bulges in blood vessels caused by weakening of an artery or vein. Commonly aneurysms occur at the circle of Willis, located on the base of the brain and in the aorta, although they can occur in other places. Bursting of the blood vessel causes death. Based on appearance, aneurysms appear either as a small bubble (like grapes) emerging from the side, these are called saccular aneurysms, or as an entire expansion of the whole circumference, making it appear like a football, these are called fusiform aneurysms.

Dissecting aneurysms (aortic dissection) are characterized by tearing off of the intimal layer of the blood vessel and subsequent formation of a hematoma in the area where the intima was. The hematoma may cover significant portions of the lumen of the blood vessel resulting in obstruction of blood flow.

The only therapeutic intervention for aneurysms and aortic dissection is surgical, which is associated with significant risk. Accordingly there is a need in the art for non-surgical methods of treating vascular degeneration and specific consequences of vascular degeneration such as aortic dissection and aneurysms.

### SUMMARY

Embodiments herein are directed to methods of inhibiting and/or reversing the process of blood vessel degeneration comprising: administering a therapeutically effective amount of a stem cell population to a degenerated blood vessel population.

Additionally, 1 or more pharmaceutical agents can also be administered, said agent capable of performing a function selected from the group consisting of: a) stimulating stem cell integration into parts of the blood vessels, b) augmenting activity of stem cells, whether endogenous or exogenous, c) an agent capable of mobilizing stem cells, and d) an agent capable of stimulating smooth muscle cell proliferation; and e) an agent inductive of nitric oxide activity.

Methods herein can include those wherein the stem cell population is selected from the group consisting of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

Pharmaceutical agents capable of stimulating stem cell integration into parts of blood vessels can be selected from the group consisting of: a) a matrix metalloprotease inhibitor, b) an antioxidant, and c) a chemoattractant.

Agents capable of stimulating stem cell activity can be selected from the group consisting of: erythropoietin, human chorionic gonadotrophin, parathyroid hormone, G-CSF, GM-CSF, valproic acid, thalidomide, and sodium phenybutyrate.

Further agents capable of mobilizing stem cells can be selected from the group consisting of: G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1.

Mobilization can be achieved by a procedure selected from the group consisting of: exercise, hyperbaric oxygen, autohemotherapy by ex vivo ozonation of peripheral blood, and induction of SDF-1 secretion in an anatomical area outside of the bone marrow.

Further agents capable of stimulating smooth muscle proliferation can be selected from the group consisting of: PDGF-1, PDGF-BB, BTC-GF, and estradiol.

Agents inductive of nitric oxide activity can be selected from the group consisting of: lipoteichoic acid, cinnamic acid, resveratrol, and FGF. Stem cells used with the teachings herein can be selected from the group consisting of: autologous, allogeneic, and xenogeneic.

Stem cells can be administered to a recipient in need and can be derived from a donor of younger age than the recipient.

Additional methods herein include treating an aneurysm in a patient in need comprising: by administering a therapeutic amount of a stem cell capable of inducing significant reversal of blood vessel degeneration.

In certain embodiments, stem cell therapy can involve intravenous administration of approximately 1-300 million CD34 stem cells and 1-300 million mesenchymal stem cells. Furthermore, stem cells can be administered once every other day for the period of a week.

Stem cells used with the methods herein can be selected from the group consisting of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells, and side population stem cells.

One or more activators of stem cells can also be administered with the methods herein, and said one or more activator can be selected from the group consisting of: erythropoietin, human chorionic gonadotrophin, parathyroid hormone, G-CSF, GM-CSF, valproic acid, thalidomide, and sodium phenybutyrate.

The administration of stem cells herein can be performed by mobilization of endogenous stem cells, said mobilization is achieved by administration of an agent selected from the group consisting of: G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA reductase inhibitors, and small molecule antagonists of SDF-1.

Mobilization herein can be achieved by a procedure selected from the group consisting of: exercise, hyperbaric oxygen, autohemotherapy by ex vivo ozonation of peripheral blood, and induction of SDF-1 secretion in an anatomical area outside of the bone marrow.

In specific embodiments, mesenchymal or mesenchymal-like stem cell populations can be administered at a concentration ranging from 500,000 to 200 million intravenously in a patient in need thereof.

In further embodiments, mesenchymal or mesenchymal-like stem cell population can express the markers CD90 and CD105 and lack expression of CD34 and CD45. Mesenchymal or mesenchymal-like stem cell population can be derived from a source selected from the group consisting of: cord blood, placenta, wharton's jelly, circulating peripheral blood, adipose tissue derived, exfoliated teeth, hair follicle, dermis, menstrual blood, endometrium, amnion, and amniotic fluid.

Furthermore, a CD34 positive stem cell population can be administered to the patient in conjunction with said mesenchymal or mesenchymal-like stem cell population.

### DETAILED DESCRIPTION

Embodiments of the present invention are described below. It is, however, expressly noted that the present invention is not limited to these embodiments, but rather the intention is that modifications that are apparent to the person skilled in the art and equivalents thereof are also included.

The invention provides methods of ameliorating, inhibiting progression of, and/or reversing blood vessel degeneration through administration of stem cells. Specifically, the invention provides the unexpected ability of systemically administered stem cells to benefit vascular function.

In one aspect of the invention a method of inducing regression of an aortic aneurysm through administration of a stem cell population, said population administered at a sufficient concentration, frequency and type so as to induce reduction in circumference of an aneurysmic blood vessel. In one embodiment, said stem cell is a mesenchymal or mesenchymal-like stem cell population that is administered at a concentration ranging from 500,000 to 200 million intravenously in a patient in need thereof. Definition of said mesenchymal or mesenchymal-like population in one embodiment includes a cell population that expresses the markers CD90, and CD105 and lacks expression of CD34 and CD45. In another embodiment positive expression of CD90 and CD105 is defined as >90% as detected by flow cytometry, and negative expression of CD34 and CD45 is defined as <10% by flow cytometry. Said mesenchymal and mesenchymal-like stem cells may be isolated from a source comprising of cord blood, placenta, wharton's jelly, circulating peripheral blood, adipose tissue derived, exfoliated teeth, hair follicle, dermis, menstrual blood, endometrium, amnion, and amniotic fluid. In another embodiment, said stem cell administered for treatment of aneurysm is a CD34 positive stem cell population that is administered alone, or in conjunction with said mesenchymal or mesenchymal-like stem cell population.

In another aspect, a method of inhibiting progression of a saccular aneurysm through administration of a stem cell population at a sufficient concentration, frequency and type so as to suppress the progress of blood vessel weakening. Said stem cell population may be a mesenchymal or mesenchymal-like stem cell population administered at a concentration ranging from 500,000 to 200 million intravenously in a patient in need thereof. Definition of said mesenchymal or mesenchymal-like population in one embodiment includes a cell population that expresses the markers CD90, and CD105 and lacks expression of CD34 and CD45. In another embodiment positive expression of CD90 and CD105 is defined as >90% as detected by flow cytometry, and negative expression of CD34 and CD45 is defined as <10% by flow cytometry. Said mesenchymal and mesenchymal-like stem cells may be isolated from a source comprising of cord blood, placenta, wharton's jelly, circulating peripheral blood, adipose tissue derived, exfoliated teeth, hair follicle, dermis, menstrual blood, endometrium, amnion, and amniotic fluid. In another embodiment, said stem cell administered for treatment of aneurysm is a CD34 positive stem cell population that is administered alone, or in conjunction with said mesenchymal or mesenchymal-like stem cell population.

In another aspect, a method of inducing regression of a saccular aneurysm through administration of a stem cell population at a sufficient concentration, frequency and type so as to induce reduction in the circumference of the saccular bulge of said aneurysm. Said stem cell population may be a mesenchymal or mesenchymal-like stem cell population administered at a concentration ranging from 500,000 to 200 million intravenously in a patient in need thereof. Definition of said mesenchymal or mesenchymal-like population in one embodiment includes a cell population that expresses the markers CD90, and CD105 and lacks expression of CD34 and CD45. In another embodiment positive expression of CD90 and CD105 is defined as >90% as detected by flow cytometry, and negative expression of CD34 and CD45 is defined as <10% by flow cytometry. Said mesenchymal and mesenchymal-like stem cells may be isolated from a source comprising of cord blood, placenta, wharton's jelly, circulating peripheral blood, adipose tissue derived, exfoliated teeth, hair follicle, dermis, menstrual blood, endometrium, amnion, and amniotic fluid. In another embodiment, said stem cell administered for treatment of aneurysm is a CD34 positive stem cell population that is administered alone, or in conjunction with said mesenchymal or mesenchymal-like stem cell population.

In another aspect of the invention, a method of inhibiting development of aortic dissection through administration of a stem cell population at a sufficient concentration, frequency and type so as to strengthen the aorta intimal layer and reduce probability of tears in said intimal layer. Said stem cell population may be a mesenchymal or mesenchymal-like stem cell population administered at a concentration ranging from 500,000 to 200 million intravenously in a patient in need thereof. Definition of said mesenchymal or mesenchymal-like population in one embodiment includes a cell population that expresses the markers CD90, and CD105 and lacks expression of CD34 and CD45. In another embodiment positive expression of CD90 and CD105 is defined as >90% as detected by flow cytometry, and negative expression of CD34 and CD45 is defined as <10% by flow cytometry. Said mesenchymal and mesenchymal-like stem cells may be isolated from a source comprising of cord blood, placenta, wharton's jelly, circulating peripheral blood, adipose tissue derived, exfoliated teeth, hair follicle, dermis, menstrual blood, endometrium, amnion, and amniotic fluid. In another embodiment, said stem cell administered for treatment of aneurysm is a CD34 positive stem cell population that is administered alone, or in conjunction with said mesenchymal or mesenchymal-like stem cell population. One method of titrating amount and type of stem cells needed is assessment of impacted on reduction of accumulation of basophilic ground substance and extent of medial cystic necrosis.

In another aspect, a method of reversing blood flow abnormalities associated with aortic dissection through administration of a stem cell population at a sufficient concentration, frequency and type so as to restore substantially normal blood flow is disclosed. Said stem cell population may be a mesenchymal or mesenchymal-like stem cell population administered at a concentration ranging from 500,000 to 200 million intravenously in a patient in need thereof. Definition of said mesenchymal or mesenchymal-like population in one embodiment includes a cell population that expresses the markers CD90, and CD105 and lacks expression of CD34 and CD45. In another embodiment positive expression of CD90 and CD105 is defined as >90% as detected by flow cytometry, and negative expression of CD34 and CD45 is defined as <10% by flow cytometry. Said mesenchymal and mesenchymal-like stem cells may be isolated from a source comprising of cord blood, placenta, wharton's jelly, circulating peripheral blood, adipose tissue derived, exfoliated teeth, hair follicle, dermis, menstrual blood, endometrium, amnion, and amniotic fluid. In another embodiment, said stem cell administered for treatment of aneurysm is a CD34 positive stem cell population that is administered alone, or in conjunction with said mesenchymal or mesenchymal-like stem cell population.

In specific embodiments of the invention, stem cell therapy is used for impeding progression of vascular aneurysms. Previous studies have demonstrated matrix metalloproteases (MMP) are involved in dilation of vascular aneurysms (13). In fact, MMP inhibitors have been proposed for clinical trials in patients with abdominal aortic aneurysms (14). Studies have also demonstrated that various stem cells including hematopoietic (15, 16) and mesenchymal (17) express high levels of MMPs. Therefore it seems counterintuitive that administration of a stem cell type would lead to regression of blood vessel disorders such as aneurysms instead of progression.

In one embodiment of the invention, hematopoietic stem cells are administered systemically into a recipient with weakened blood vessels. Said hematopoietic stem cells may be extracted from sources known in the art such as cord blood, peripheral blood, mobilized peripheral blood, and bone marrow. Additionally hematopoietic stem cells may by generated in vitro by differentiation from embryonic stem cells or other precursor populations. For the practice of the current invention hematopoietic stem cells may be autologous or allogeneic. If allogeneic cells are used, steps to remove immunogenic components may be taken. For example, hematopoietic stem cells may be purified substantially of contaminating leukocytes. Said purification procedures are known in the art and include selection for markers associated with hematopoietic stem cells such as CD34 and/or CD133.

In some embodiments matching of allogeneic hematopoietic stem cells may be accomplished by use of HLA typing or using procedures such as mixed lymphocyte reaction as previously described in the patent application PCT/US2007/020415 entitled Allogeneic Stem Cell Transplants in Non-conditioned Recipients.

In one aspect of the invention, blood vessel function may be restored by administration of a group of cells, said group comprising of: stem cells, committed progenitor cells, and differentiated cells. Said stem cells may be selected from a group comprising of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells. In some aspects of the invention, embryonic stem cells are totipotent and may express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT). Non-embryonic stem cells may be derived from cord blood stem cells possess multipotent properties and are capable of differentiating into endothelial, smooth muscle, and neuronal cells. Cord blood stem cells useful for the practice of the invention may be identified based on expression of one or more antigens selected from a group comprising: SSEA-3, SSEA-4, CD9, CD34, c-kit, OCT-4, Nanog, and CXCR-4, additionally, cord blood stem cells do not express one or more markers selected from a group comprising of: CD3, CD34, CD45, and CD11b.

In another aspect of the invention, placental stem cells are isolated from the placental structure and administered for the purpose of regeneration of blood vessel function. Said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

In another aspect of the invention, bone marrow stem cells are isolated from the bone marrow and administered for regeneration of blood vessel function. Said bone marrow stem cells may be bone marrow derived mononuclear cells, said mononuclear cells containing populations capable of differentiating into one or more of the following cell types: endothelial cells, smooth muscle cells, and neuronal cells. In one embodiment, said bone marrow stem cells may be selected based on expression of one or more of the following antigens: CD34, c-kit, flk-1, Stro-1, CD105, CD73, CD31, CD146, vascular endothelial-cadherin, CD133 and CXCR-4. Additionally, stem cell activity may be enhanced by selecting for cells expressing the marker CD133.

In another aspect of the invention, stem cells may be isolated from amniotic fluid and used for regeneration of blood vessel function. Said isolation may be accomplished by purifying mononuclear cells, and/or c-kit expressing cells from amniotic fluid, said fluid may be extracted by means known to one of skill in the art, including utilization of ultrasound guidance. Said amniotic fluid stem cells may be selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1 or lack of significant expression of one or more of the following antigens: CD34, CD45, and HLA Class II.

In another aspect of the invention, neuronal stem cells may be utilized as a cell source capable of regeneration of blood vessel function. Said neuronal stem cells are selected based on expression of one or more of the following antigens: RC-2, 3CB2, BLB, Sox-2hh, GLAST, Pax 6, nestin, Muashi-1, NCAM, A2B5 and prominin.

In another aspect of the invention, circulating peripheral blood stem cells are utilized for regeneration of blood vessel function. Said peripheral blood stem cells are characterized by ability to proliferate in vitro for a period of over 3 month and by expression of CD34, CXCR4, CD117, CD113, and c-met, and lack of differentiation associated markers, said markers may be selected from a group comprising of CD2, CD3, CD4, CD11, CD11a, Mac-1, CD14, CD16, CD19, CD24, CD33, CD36, CD38, CD45, CD56, CD64, CD68, CD86, CD66b, and HLA-DR.

In another aspect of the invention mesenchymal stem cells are utilized for regeneration of blood vessel function. Said mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and do not express substantial levels of HLA-DR, CD117, and CD45. Said mesenchymal stem cells are derived from a group selected of: bone marrow, adipose tissue, endometrium, menstrual blood, umbilical cord blood, placental tissue, peripheral blood mononuclear cells, differentiated embryonic stem cells, and differentiated progenitor cells.

In another aspect of the invention germinal stem cells are utilized for regeneration of blood vessel function, said cells express markers selected from a group comprising of: Oct4, Nanog, Dppa5 Rbm, cyclin A2, Tex18, Stra8, Dazl, beta1- and alpha6-integrins, Vasa, Fragilis, Nobox, c-Kit, Sca-1 and Rex1.

In another aspect of the invention adipose tissue derived stem cells are utilized for regeneration of blood vessel function, wherein said adipose tissue derived stem cells express markers selected from a group comprising of: CD13, CD29, CD44, CD63, CD73, CD90, CD166, Aldehyde dehydrogenase (ALDH), and ABCG2, and said adipose tissue derived stem cells are a population of purified mononuclear cells extracted from adipose tissue capable of proliferating in culture for more than 1 month.

In another aspect of the invention exfoliated teeth derived stem cells are utilized for regeneration of blood vessel function, wherein said exfoliated teeth derived stem cells express markers selected from a group comprising of: STRO-1, CD146 (MUC18), alkaline phosphatase, MEPE, and bFGF.

In another aspect of the invention hair follicle stem cells are utilized for regeneration of blood vessel function, wherein said cells express markers selected from a group comprising of: cytokeratin 15, Nanog, and Oct-4, and, wherein said hair follicle stem cells are capable of proliferating in culture for a period of at least one month, and wherein said hair follicle stem cells secrete one or more of the following proteins when grown in culture: basic fibroblast growth factor (bFGF), endothelin-1 (ET-1) and stem cell factor (SCF).

In another aspect of the invention dermal stem cells are utilized for regeneration of blood vessel function, wherein said cells express markers selected from a group comprising of: CD44, CD13, CD29, CD90, and CD105 and are capable of proliferating in culture for a period of at least one month

In another aspect of the invention parthenogenically derived stem cells are utilized for regeneration of blood vessel function, said parthenogenically derived stem cells may be generated by addition of a calcium flux inducing agent to activate an oocyte followed by enrichment of cells expressing markers selected from a group comprising of SSEA-4, TRA 1-60 and TRA 1-81.

### Example 1

50 patients with an abnormal dilatation of the abdominal aorta (average circumference 3.5 cm) are entered into a clinical trial. 25 are treated with placebo, and 25 receive stem cells (active treatment). The stem cell group receives 5 million CD34 cells derived from cord blood are given intravenously and 3 million mesenchymal stem cells are given intravenously. Cells are given for 4 consecutive days. On day 7 cells are administered again, 5 million CD34 and 3 million mesenchymal cells. After a period of 16 weeks the average circumference of the aorta at the point of dilation is 4.7 cm in the placebo group, whereas in the active treatment group the average circumference is 3.2 cm.

Mesenchymal cells are prepared as described in as described in Meng et al. Endometrial regenerative cells: a novel stem cell population. J Transl Med. 2007 Nov. 15; 5:57). CD34 cells are extracted and expanded as described below

Umbilical cord blood is purified according to routine methods ((Rubinstein, et al. Processing and cryopreservation of placental/umbilical cord blood for unrelated bone marrow reconstitution. Proc Natl Acad Sci USA 92:10119-10122). Briefly, a 16-gauge needle from a standard Baxter 450-ml blood donor set containing CPD A anticoagulant (citrate/phosphate/dextrose/adenine) (Baxter Health Care, Deerfield, Ill.) is inserted and used to puncture the umbilical vein of a placenta obtained from healthy delivery from a mother tested for viral and bacterial infections according to international donor standards. Cord blood is allowed to drain by gravity so as to drip into the blood bag. The placenta is placed in a plastic-lined, absorbent cotton pad suspended from a specially constructed support frame in order to allow collection and reduce the contamination with maternal blood and other secretions, The 63 ml of CPD A used in the standard blood transfusion bag, calculated for 450 ml of blood, is reduced to 23 ml by draining 40 ml into a graduated cylinder just prior to collection. This volume of anticoagulant matches better the cord volumes usually retrieved (<170 ml).

An aliquot of the blood is removed for safety testing according to the standards of the National Marrow Donor Program (NMDP) guidelines. Safety testing includes routine laboratory detection of human immunodeficiency virus 1 and 2, human T-cell lymphotropic virus I and II, Hepatitis B virus, Hepatitis C virus, Cytomegalovirus and Syphilis. Subsequently, 6% (wt/vol) hydroxyethyl starch is added to the anticoagulated cord blood to a final concentration of 1.2%. The leukocyte rich supernatant is then separated by centrifuging the cord blood hydroxyethyl starch mixture in the original collection blood bag (50×g for 5 min at 10° C.). The leukocyte-rich supernatant is expressed from the bag into a 150-ml Plasma Transfer bag (Baxter Health Care) and centrifuged (400×g for 10 min) to sediment the cells. Surplus supernatant plasma is transferred into a second plasma Transfer bag without severing the connecting tube. Finally, the sedimented leukocytes are resuspended in supernatant plasma to a total volume of 20 ml. Approximately 5×10⁸-7×10⁹ nucleated cells are obtained per cord. Cells are cryopreserved according to the method described by Rubinstein et al (Rubinstein, et al. Processing and cryopreservation of placental/umbilical cord blood for unrelated bone marrow reconstitution. Proc Natl Acad Sci USA 92:10119-10122). for subsequent cellular therapy. CD34 cells are expanded by culture. CD34+ cells are purified from the mononuclear cell fraction by immuno-magnetic separation using the Magnetic Activated Cell Sorting (MACS) CD34+ Progenitor Cell Isolation Kit (Miltenyi-Biotec, Auburn, Calif.) according to manufacturer's recommendations. The purity of the CD34+ cells obtained ranges between 95% and 98%, based on Flow Cytometry evaluation (FACScan flow cytometer, Becton-Dickinson, Immunofluorometry systems, Mountain View, Calif.). Cells are plated at a concentration of 10.sup.4 cells/ml in a final volume of 0.5 ml in 24 well culture plates (Falcon; Becton Dickinson Biosciences) in DMEM supplemented with the cytokine cocktail of: 20 ng/ml IL-3, 250 ng/ml IL-6, 10 ng/ml SCF, 250 ng/ml TPO and 100 ng/ml flt-3L and a 50% mixture of LPCM. LPCM is generated by obtaining a fresh human placenta from vaginal delivery and placing it in a sterile plastic container. The placenta is rinsed with an anticoagulant solution comprising phosphate buffered saline (Gibco-Invitrogen, Grand Island, N.Y.), containing a 1:1000 concentration of heparin (1% w/w) (American Pharmaceutical Partners, Schaumburg, Ill.). The placenta is then covered with a DMEM media (Gibco) in a sterile container such that the entirety of the placenta is submerged in said media, and incubated at 37.degree. C. in a humidified 5% CO.sub.2 incubator for 24 hours. At the end of the 24 hours, the live placenta conditioned medium (LPCM) is isolated from the container and sterile-filtered using a commercially available sterile 0.2 micron filter (VWR). Cells are expanded, checked for purity using CD34-specific flow cytometry and immunologically matched to recipients using a mixed lymphocyte reaction. Cells eliciting a low level of allostimulatory activity to recipient lymphocytes are selected for transplantation. Cells are administered as described above.

### Example 2

60 eight week-old Fbn1(C1039G/+) mice on a BALB/c background are selected and randomized into 2 groups of 30 each. The first group received intravenous administration of 500,000 BALB/c derived bone marrow mesenchymal stem cells. The second group receives 500,000 BALB/c splenocytes as a control. Administration of cells is performed weekly for the period of 4 weeks. Evaluation of spontaneous aortic degeneration is performed as described in Chung et al. (Long-term doxycycline is more effective than atenolol to prevent thoracic aortic aneurysm in marfan syndrome through the inhibition of matrix metalloproteinase-2 and -9. Circ Res. 2008 Apr. 25; 102(8):e73-85). Briefly, aortic segments are collected from 10 mice each at 3, 6, and 9 months. Vessel strength, elastic fiber composition, and aortic stiffness are assessed. Mice which received mesenchymal stem cells demonstrated increased vessel strength, elastic fiber content, and aortic stiffness.

### Example 3

60 eight week-old rats are selected and randomized into 2 groups of 30 each. Rats are administered elastase in order to induce abdominal aortic aneurysms as described (Tomita et al. Inhibition of experimental abdominal aortic aneurysm progression by nifedipine. Int J Mol Med. 2008 February; 21(2):239-44). Cell therapy was performed as follows: the first group of rats received intravenous administration of 500,000 syngeneic bone marrow mesenchymal stem cells. The second group receives syngeneic splenocytes as a control. Administration of cells is performed weekly for the period of 2 weeks subsequent to infusion of elastase. Rats were sacrificed on week 4. Decreased aortic dilation was observed in the animals receiving stem cell therapy as compared to control splenocytes.

### Example 4

A patient with an aortic aneurysm with a circumference of 6.6 cm as determined by CT scan presented for stem cell therapy. After obtaining informed consent and explaining the experimental nature of the procedure, the patient was accepted for treatment under a compassionate-use basis.

One day 1 the patient underwent compatibility testing to determine a batch of CD34 cells useful for therapy. One day 2 the patient received 25 grams of intravenous vitamin C in a volume of 250 cc, as well as 5 million CD34 cells and 3 million endometrial regenerative cells. On days 3-5 the patient received 5 million CD34 cells and 3 million endometrial regenerative cells intravenously. On day 7 the patient received 25 grams of intravenous vitamin C and subsequently 1.5 million CD34 and 3 million endometrial derived regenerative cells.

Three weeks after treatment CT scan reports the aneurysm decreased in circumference to 5.3 cm.

Endometrial regenerative cells were prepared as described in as described in Meng et al. Endometrial regenerative cells: a novel stem cell population. J Transl Med. 2007 Nov. 15; 5:57). CD34 cells are where extracted, expanded and matched as described in Example 1.

It is understood by those of skilled in the art that the steps in the above method can be practiced in various different orders. The listing of the steps in the particular order described above does not, and should not, limit the disclosed method to the particular disclosed order of steps.

The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

### REFERENCES

The following is a list of references cited herein, each of which is expressly incorporated by reference in their entireties:
1. Sagripanti, A., and Carpi, A. 2000. Antithrombotic and prothrombotic activities of the vascular endothelium. Biomed Pharmacother 54:107-111.
2. Correia, M. L., and Haynes, W. G. 2007. Arterial compliance and endothelial function. Curr Diab Rep 7:269-275.
3. Stefano, G. B., Christensen, V. B., Tonnesen, E., Liu, Y., Hughes, T. K., Jr., and Bilfinger, T. V. 1997. Interleukin-10 stimulation of endogenous nitric oxide release from human saphenous veins diminishes immunocyte adherence. J Cardiovasc Pharmacol 30:90-95.
4. Hristov, M., Zernecke, A., Liehn, E. A., and Weber, C. 2007. Regulation of endothelial progenitor cell homing after arterial injury. Thromb Haemost 98:274-277.
5. Regnault, V., and Levy, B. 2007. Recombinant activated protein C in sepsis: endothelium protection or endothelium therapy? Crit Care 11:103.
6. Yavuz, B. B., Yavuz, B., Sener, D. D., Cankurtaran, M., Halil, M., Ulger, Z., Nazli, N., Kabakci, G., Aytemir, K., Tokgozoglu, L., et al. 2008. Advanced Age Is Associated with Endothelial Dysfunction in Healthy Elderly Subjects. Gerontology.
7. Lauer, T., Heiss, C., Balzer, J., Kehmeier, E., Mangold, S., Leyendecker, T., Rottler, J., Meyer, C., Merx, M. W., Kelm, M., et al. 2008. Age-dependent endothelial dysfunction is associated with failure to increase plasma nitrite in response to exercise. Basic Res Cardiol 103:291-297.
8. Hurme, M., Kivimaki, M., Pertovaara, M., Lehtimaki, T., Karhunen, P. J., Jylha, M., Hervonen, A., and Eklund, C. 2007. CRP gene is involved in the regulation of human longevity: a follow-up study in Finnish nonagenarians. Mech Ageing Dev 128:574-576.
9. Ferri, C., Croce, G., Cofini, V., De Berardinis, G., Grassi, D., Casale, R., Properzi, G., and Desideri, G. 2007. C-reactive protein: interaction with the vascular endothelium and possible role in human atherosclerosis. Curr Pharm Des 13:1631-1645.
10. Teoh, H., Quan, A., Lovren, F., Wang, G., Tirgari, S., Szmitko, P. E., Szalai, A. J., Ward, M. E., and Verma, S. 2008. Impaired endothelial function in C-reactive protein overexpressing mice. Atherosclerosis.
11. Hristov, M., Zernecke, A., Bidzhekov, K., Liehn, E. A., Shagdarsuren, E., Ludwig, A., and Weber, C. 2007. Importance of CXC chemokine receptor 2 in the homing of human peripheral blood endothelial progenitor cells to sites of arterial injury. Circ Res 100:590-597.
12. Kondo, S., Hashimoto, N., Kikuchi, H., Hazama, F., Nagata, I., and Kataoka, H. 1998. Apoptosis of medial smooth muscle cells in the development of saccular cerebral aneurysms in rats. Stroke 29:181-188; discussion 189.
13. Sangiorgi, G., D′Averio, R., Mauriello, A., Bondio, M., Pontillo, M., Castelvecchio, S., Trimarchi, S., Tolva, V., Nano, G., Rampoldi, V., et al. 2001. Plasma levels of metalloproteinases-3 and -9 as markers of successful abdominal aortic aneurysm exclusion after endovascular graft treatment. Circulation 104:I288-295.
14. Thompson, R. W., and Baxter, B. T. 1999. MMP inhibition in abdominal aortic aneurysms. Rationale for a prospective randomized clinical trial. Ann N Y Acad Sci 878:159-178.
15. Yu, X. F., and Han, Z. C. 2006. Matrix metalloproteinases in bone marrow: roles of gelatinases in physiological hematopoiesis and hematopoietic malignancies. Histol Histopathol 21:519-531.
16. Rao, Q., Zheng, G. G., Lin, Y. M., and Wu, K. F. 2004. Production of matrix metalloproteinase-9 by cord blood CD34+ cells and its role in migration. Ann Hematol 83:409-413.
17. Ghajar, C. M., Blevins, K. S., Hughes, C. C., George, S. C., and Putnam, A. J. 2006. Mesenchymal stem cells enhance angiogenesis in mechanically viable prevascularized tissues via early matrix metalloproteinase upregulation. Tissue Eng 12:2875-2888.

1. A method of inhibiting and/or reversing the process of blood vessel degeneration comprising: administering a therapeutically effective amount of a stem cell population to a degenerated blood vessel population.

2. The method of claim 1, wherein a pharmaceutical agent is also administered, said agent capable of performing a function selected from the group consisting of: a) stimulating stem cell integration into parts of the blood vessels, b) augmenting activity of stem cells, whether endogenous or exogenous, c) an agent capable of mobilizing stem cells, and d) an agent capable of stimulating smooth muscle cell proliferation; and e) an agent inductive of nitric oxide activity.

3. The method of claim 1, wherein said stem cell population is selected from the group consisting of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

4. The method of claim 2, wherein said pharmaceutical agent stimulating stem cell integration into parts of blood vessels is selected from the group consisting of:
a) a matrix metalloprotease inhibitor, b) an antioxidant, and c) a chemoattractant.

5. The method of claim 2, wherein said agent capable of stimulating stem cell activity is selected from the group consisting of: erythropoietin, human chorionic gonadotrophin, parathyroid hormone, G-CSF, GM-CSF, valproic acid, thalidomide, and sodium phenybutyrate.

6. The method of claim 2, wherein said agent capable of mobilizing stem cells is selected from the group consisting of: G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1.

7. The method of claim 2, wherein said mobilization is achieved by a procedure selected from the group consisting of: exercise, hyperbaric oxygen, autohemotherapy by ex vivo ozonation of peripheral blood, and induction of SDF-1 secretion in an anatomical area outside of the bone marrow.

8. The method of claim 2, wherein said agent capable of stimulating smooth muscle proliferation is selected from the group consisting of: PDGF-1, PDGF-BB, BTC-GF, and estradiol.

9. The method of claim 2, wherein said agent inductive of nitric oxide activity is selected from the group consisting of: lipoteichoic acid, cinnamic acid, resveratrol, and FGF.

10. The method of claim 2 wherein said stem cells are selected from the group consisting of: autologous, allogeneic, and xenogeneic.

11. The method of claim 2, wherein said stem cells are administered to a recipient in need and are derived from a donor of younger age than the recipient.

12. A method of treating an aneurysm in a patient in need comprising: administering a therapeutic amount of a stem cell capable of inducing significant reversal of blood vessel degeneration.

13. The method of claim 12 wherein said stem cell therapy involves intravenous administration of approximately 1-300 million CD34 stem cells and 1-300 million mesenchymal stem cells.

14. The method claim 13, wherein said cells are administered once every other day for the period of a week.

15. The method of claim 12, wherein said stem cell is selected from the group consisting of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells, and side population stem cells.

16. The method of claim 12, wherein an activator of stem cells is also administered, and said activator is selected from the group consisting of: erythropoietin, human chorionic gonadotrophin, parathyroid hormone, G-CSF, GM-CSF, valproic acid, thalidomide, and sodium phenybutyrate.

17. The method of claim 12, wherein said administration of stem cell is performed by mobilization of endogenous stem cells, said mobilization is achieved by administration of an agent selected from the group consisting of: G-CSF, M-CSF, GM-CSF, 5-FU, IL-1, IL-3, hyaluronic acid fragments, kit-L, VEGF, Flt-3 ligand, PDGF, EGF, FGF-1, FGF-2, TPO, IL-11, IGF-1, MGDF, NGF, HMG CoA) reductase inhibitors, and small molecule antagonists of SDF-1.

18. The method of claim 17, wherein said mobilization is achieved by a procedure selected from the group consisting of: exercise, hyperbaric oxygen, autohemotherapy by ex vivo ozonation of peripheral blood, and induction of SDF-1 secretion in an anatomical area outside of the bone marrow.

19. The method of claim 12, wherein a mesenchymal or mesenchymal-like stem cell population is administered at a concentration ranging from 500,000 to 200 million intravenously in a patient in need thereof.

20. The method of claim 19, wherein said mesenchymal or mesenchymal-like stem cell population expresses the markers CD90 and CD105 and lacks expression of CD34 and CD45.

21. The method of claim 20, wherein said mesenchymal or mesenchymal-like stem cell population is from a source selected from the group consisting of: cord blood, placenta, wharton's jelly, circulating peripheral blood, adipose tissue derived, exfoliated teeth, hair follicle, dermis, menstrual blood, endometrium, amnion, and amniotic fluid.

22. The method of claim 19, wherein a CD34 positive stem cell population is administered to the patient in conjunction with said mesenchymal or mesenchymal-like stem cell population.
